(19) United States
(12) Patent Application Publication
(10) Pub. No.: US 2009/0203897 A1
(43) Pub. Date: Aug. 13, 2009

(54) Method of Using Polymer Embedded Solid Supports for Small Scale Oligonucleotide Synthesis

(75) Inventors: Lance Larka, Huntsville, AL (US); Philipp Wenter, Huntsville, AL (US); Patrick A. Weiss, Huntsville, AL (US)

Correspondence Address: LANIER FORD SHAVER & PAYNE P.C., P O BOX 2087, HUNTSVILLE, AL, 35804-2087, US

(73) Assignee: Operon Biotechnologies, Inc., Huntsville, AL (US)

(21) Appl. No.: 12/028,083

(22) Filed: Feb. 8, 2008

(51) Int. Cl. C07H 21/00

(52) U.S. Cl. 536/25.34; 536/25.3

(57) ABSTRACT

A method of synthesizing oligonucleotides is claimed. The method utilizes a synthesis column incorporating a filter-like porous polymer cartridge which contains derivatized Controlled Pore Glass (CPG) or cross-linked Polystyrene (PS) directly embedded into the polymer cartridge. Small-scale synthesis of oligonucleotides of a predetermined sequence may be accomplished by introducing reagents of the phosphoramidite method into the synthesis column. These reagents flow through the cartridge allowing the reaction sequence to take place on the derivatized CPG or PS incorporated within the polymer cartridge.

### FIELD OF THE DISCLOSURE

The present disclosure relates generally to oligonucleotide synthesis. In particular, the present disclosure relates to solid phase oligonucleotide synthesis on a hybrid material consisting of conventional oligonucleotide solid supports embedded into a porous polymer matrix. More particularly, the present disclosure relates to using derivatized Control Pore Glass (CPG) or derivatized cross-linked Polystyrene (PS) embedded into porous plastics such as Polyethylen (PE) as a solid supports for small scale oligonucleotide synthesis.

### BACKGROUND OF THE DISCLOSURE

Oligonucleotides are short strands of DNA or RNA, typically with a length of 4-100 nucleotides. DNA consists of the four deoxyribo-nucleotides: deoxy-adenosine (dA), deoxy-cytosine (dC), deoxy-guanosine (dG), and thymine (T). Modern biotechnology requires short DNA oligonucleotides as an essential component of many applications including Polymerase Chain Reaction (PCR), gene sequencing, hybridization gel shift assays, cloning, the generation of genetic libraries (CDNA libraries), mutagenesis, antisense technology, and gene synthesis. RNA consists of the of the four ribo-nucleotides: adenosine (A), cytosine (C), guanosine (G) and uridien (U). Important applications in biotechnology include gene silencing through RNA interference, which uses synthetic short double stranded RNAs (siRNA) to switch off specific genes of living organisms.

Most of the oligonucleotides employed for these applications are prepared chemically by solid phase synthesis. Chemical solid phase synthesis is a fast, efficient and highly flexible synthesis method that allows the production and delivery of customized oligonucleotides within days. Chemical solid phase synthesis is usually carried out on solid supports such as Controlled Pore Glass (CPG) or cross-linked Polystyrene (PS). Both solid supports are composed of very fine particles in the μm-range and similar in appearance and characteristics to fine grained sea sand. They contain pores of a defined size which are usually either 500 A or 1000 A. To serve as a solid support for oligonucleotide synthesis, CPG or cross-linked PS may be activated and derivatized with either dA, dC, dG or T nucleotides. Alternatively, the solid support may be derivatized with a universal linker suitable for oligonucleotide synthesis. Accordingly, the first nucleotide of the oligonucleotide chain is present on the solid support at the initiation of synthesis. Alternatively, the solid support may be derivatized with universal linkers which allow any type of oligonucleotide to be synthesized regardless of type of nucleotide present at the 3′-end. Typical loadings are 20 to 40 μmol/g for CPG and 40 to 200 μmol/g for cross-linked PS.

Oligonucleotides are synthetized on the solid support in a step-by-step addition of one nucleotide after the other. The standard method used in this context is the so called phosphoramidite method. Each addition of a nucleotide requires a total of four chemical reactions: Release of the 5′-OH (deblocking of the 5′-OH protecting group), coupling of the desired nucleotide in its phosphoramidite form, capping of unreacted 5′-OH positions, and oxidation. This reaction sequence is called a coupling cycle. It is repeated for addition of further nucleotides until the desired sequence length is reached. At synthesis end, the resulting oligonucleotide is cleaved from the solid support, deprotected and collected in solution.

Oligonucleotide solid phase synthesis can be carried out by automated synthesis using oligonucleotide synthesizers. Automation allows the preparation of oligonucleotides with fast turn-around and high throughput. Modern high throughput industrial synthesizers allow hundreds of oligonucleotides to be synthesized in parallel by using assemblies of synthesis columns. Synthesis columns are typically thin, cylindrical tubes containing a derivatized solid support disposed between two porous plastic fits. In traditional oligonucleotide synthesis, these frits act as filters and must be considered when determining reagent volume and flow rates for the synthesis process. Synthesis columns are often arranged vertically on an 8×12 plate. The reagents for the oligonucleotide synthesis are either pumped through the synthesis column or dispensed directly into the synthesis column on top of the upper frit and allowed to flow through the solid support by gravity, pressure or vacuum.

The amount of loaded solid support used for synthesis determines the volume of chemicals used and consumed during the synthesis process and the total amount of final product produced. CPG-based synthesis columns for high throughput synthesis are currently available for synthesis scales of 10 nanomole (nmol) to 10 micromole (μmol). The amount of CPG contained in the columns depends on the synthesis scale and the loading of the CPG. For a typical CPG-loading of 20 to 40 micromole/gram (μmol/g) the amount of CPG employed for different synthesis scales is listed in Table 1:

| Synthesis scale | | Amount of CPG |
|---|---|---|
| 10 | nmol* | 0.25-0.5 mg |
| 50 | nmol | 1.25-2.5 mg |
| 100 | nmol | 2.5-5 mg |
| 200 | nmol | 5-10 mg |
| 1 | μmol | 25-50 mg |
| 10 | μmol | 250-500 mg |

*10 nmol synthesis scale is the smallest commonly available scale for commercial oligonucleotide synthetizers.

Over the last decade the required quantity of oligonucleotide has decreased considerably. This is due to the fact that many applications in molecular biology only need a small amount of oligonucleotide (c.f. the DNA primers for PCR reactions). Often much less than 10 nmol is required. On the other hand the number of custom oligonucleotides needed each day has continuously increased and is manufactured in a high throughput plate based parallel synthesis process.

For high throughput oligonucleotide synthesis large numbers of synthesis columns containing accurate amounts of CPG or cross-linked PS have to be prepared. CPG and cross-linked PS are both highly electrostatic powders. This property causes severe problems for accurate dispensing of small amounts in an automated way. The lower limit for solid support portions that can still be dispensed with a high enough accuracy and reproducebility by current dispensing technologies is approximately 1-2 milligrams (mg). To scale the process down further an inexpensive, reliable method of holding a reduced amount of CPG or cross-linked PS in a reaction container suitable for mechanical automation is required.

At the current lower limit of dispensing technology, the synthesis column volume taken up by the CPG or cross-linked PS is much smaller than the volume taken up by the frits they disposed between. The chemical reaction takes place at the interface of reaction fluid and the solid support. The additional volume which is needed to soak the frits so that it can reach the solid support is called dead volume and increases the total reagents consumption without benefit for the reaction itself Reducing the dead volume of the synthesis results in a direct reduction of the reagents consumption, thereby achieving an immediate cost saving.

### SUMMARY OF THE DISCLOSURE

Applicant has addressed the need for small scale solid supports for oligonucleotide synthesis by providing a method of using derivatized CPG or cross-linked PS which is embedded into a frit or porous polymer cartridge. Current technology allows for the preparation of porous polymer cartridges from polymer granulates in variable shape and with different porosity. In order to prepare a novel and improved solid support for high throughput oligonucleotide synthesis, the Applicant has incorporated derivatized CPG or PS into High Molecular Weight Polyethylene (HMWPE) cartridges. Incorporating derivatized CPU or PS into a polymer matrix allows for smaller synthesis scales by eliminating the need to dispense small amounts of loose and highly electrostatic CPG or PS into synthesis columns. For the preparation of the small scale solid supports, derivatized CPG or PS is mixed in the desired ratio with the HMWPE polymer granulates to create a bulk mixture. The bulk mixture is then distributed into the cavities of a sintering mold. After a controlled sintering process rod-like cartridges of HMWPE containing defined amounts of CPG or PS are obtained. Incorporating CPG or PS into the polymer cartridge facilitates its handling in a high throughput industrial environment. CPG or PS is not handled in loose form but embedded into a rod-like polymer cartridge which is especially helpful for loading it into columns or assembling plates for modern high throughput and plate-based synthetizers. Using CPG or PS which is immobilized in a polymer cartridge allows a reduction in the reagent consumption for synthesis considerably because most of the dead volume is eliminated.

### BRIEF DESCRIPTION OF THE DRAWINGS

The following drawings incorporated in and forming a part of the specification illustrate, and together with the detailed description, serve to explain the various aspects of the implementation(s) and/or embodiments of the disclosure and not of the disclosure itself.

FIG. 1 is a perspective view of a synthesis column used in accordance with the prior art method.

FIG. 2 is a perspective view of a synthesis column use in accordance with the present invention.

FIG. 3 is a flowchart depicting the exemplary method of using a derivatized polymer cartridge.

FIG. 4 is a graph of High Performance Liquid Chromotography (HPLC) traces of oligonucleotides synthesized using an exemplary method of the present disclosure depicted in FIG. 3.

FIG. 5 is a graph of High Performance Liquid Chromotography (HPLC) traces of oligonucleotides synthesized using the exemplary method of the present disclosure depicted in FIG. 3.

### DETAILED DESCRIPTION

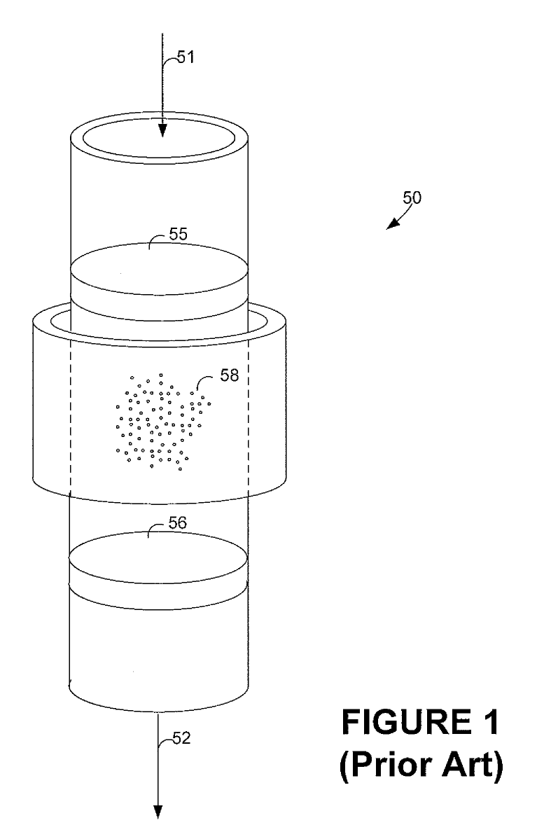

The various embodiments of the present disclosure and their advantages are best understood by referring to FIGS. 1 through 5 of the drawings. It will be apparent to those skilled in the art that various modifications and variations can be made in the present disclosure without departing from the scope and spirit of the disclosure as described herein. For instance, features illustrated or described as part of one embodiment can be included in another embodiment to yield a still further embodiment. Moreover, variations in selection of materials and/or characteristics may be practiced to satisfy particular desired user criteria. Thus, it is intended that the present disclosure covers such modifications as come within the scope of the present features and their equivalents.

FIG. 1 depicts a synthesis column 50 of the prior art. Two flits 55 and 56 are disposed within said column 50 with derivatized CPG 58 disposed between the top frit 55 and the bottom frit 56. Reagents 51 are introduced into the column 50, flow through the first frit 55, and initiate their associated chemical reaction on the derivatized CPG 58 while the reagents flow through the second frit 56, and eventually the spent reagents 52 exit the column 50.

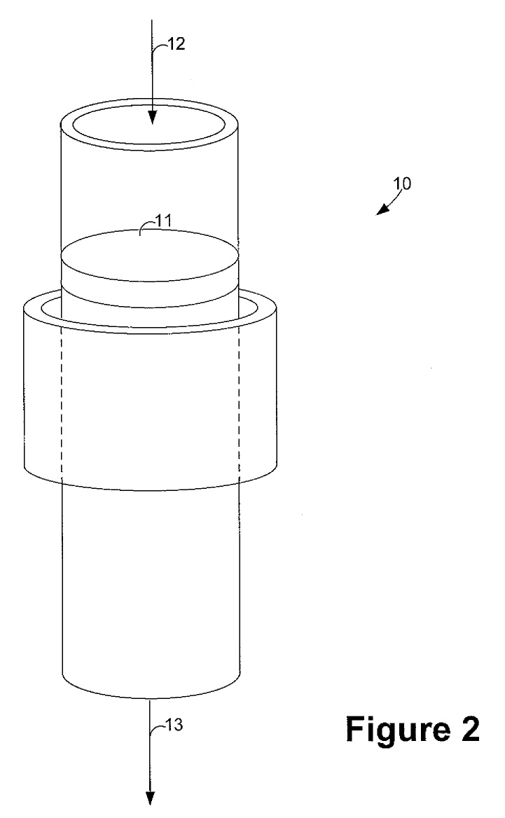

FIG. 2 depicts a synthesis column 10 of the preset disclosure. In accordance with the present invention, the synthesis column 10 is equipped a filter-like porous polymer cartridge 11 which contains the derivatized CPG or PS directly embedded into the polymer cartridge. Oligonucleotides of a predetermined sequence are synthetized by introducing reagents 12 into the synthesis column 10. Reagents 12 flow through the cartridge 11 allowing the reaction sequence to take place on the derivatized CPG or PS (not pictured) incorporated within the polymer cartridge 11, while leftover reagent 13 leaves the column 10. Reagents for the phosphoramidite method to continue the synthesis through the addition of subsequent nucleotides are well known to those skilled in the art. In general, the reagents used in the phosphoramidite method include a solution of an organic acid such as dichloro acetic acid in an appropriate organic solvent, a solution of phosphoramidite and an appropriate activator, a solution oxidating agent such iodine/water or tert-butylperoxide in an appropriate organic solvent and a solution of an organic acid anhydride such as acetic acid anhydride and an organic base in an appropriate organic solvent. Specific reagents used for the phosphoramidite method include a solution of dichloroacetic acid in dichloromethane, a solution of phosphoramidite and tetrazol activator in acetonitril, a solution of acetic acid anhydride, lutidine and 1 -methylimidazol in tetrahydrofurane, and a solution of iodine in a mixture of pyridine, tetrahydrofurane and water

Thus, the Phosphoramidite method requires a total of four chemical reactions in order to introduce each subsequent nucleotide; release of the 5′-OH (deblocking of the 5′-OH protecting group), coupling of the desired nucleotide in its phosphoramidite form, capping of unreacted 5′-OH positions, and oxidation. This reaction sequence is repeated for addition of further nucleotides. At synthesis end, the resulting oligonueleotide is cleaved from the solid support, deprotected and collected in solution.

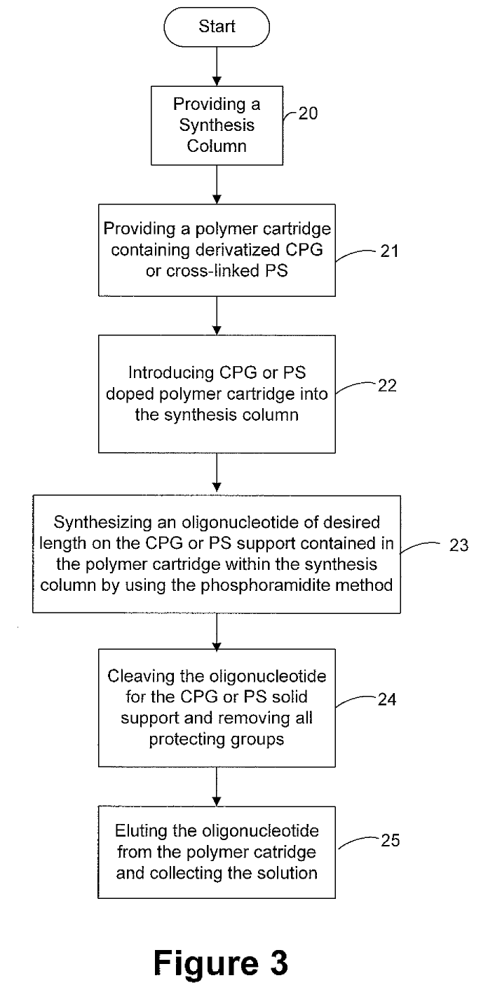

FIG. 3 is a flowchart depicting the method of synthesizing an oligonucleotide of a predetermined sequence. First, a synthesis column 10 (FIG. 2) is provided as indicated in step 20. Next, a polymer cartridge containing derivatized CPG or cross linked PS is provided as indicated in step 21. The polymer cartridge 11 (FIG. 2) is disposed within said column as indicated in step 22. Next, using the phosphoramidite method an oligonucleotide is synthetized on the CPG or PS contained within the polymer cartridge by applying the appropriate reagents into the synthesis column 10 (FIG. 2) as indicated in step 23. If the desired oligonucleotide has been formed the oligonucleotide is cleaved from the CPG- or PS-solid support and deprotected, as indicated in step 24. Subsequently the oligonucleotide is eluded from the synthesis column and collected, as indicated in step 25.

According to the present disclosure, the derivatized CPG or PS is incorporated into the porous HMWPE cartridge by mixing derivatized CPG or PS in the desired ratio with the HMWPE polymer granulates, distributing the mixture into the cavities of an appropriate mold and sintering the polymer at 160-200 C for 30 to 60 s. In alternative embodiments, the following thermoplastics may also be used in lieu of HMWPE: examples of suitable polyolefines include but are not limited to: ethylene vinyl acetate; ethylene methyl acrylate; polyethylenes; polypropylenes; ethylene-propylene rubbers; ethylene-propylenediene rubbers; poly(1-butene); polystyrene; poly(2-butene); poly(1-pentene); poly(2-pentene); ploy(3-methyl-1-pentene); poly(4-methyl-1-pentene); 1,2-poly-1,3-butadiene; 1,4-poly-1,3-butadiene; polyisoprene; polychloroprene; poly(vinyl acetate); poly(vinyldiene chloride); and mixtures and derivatives thereof. In alternative embodiments nylons, polycarbonates, poly(ether sulfones), and mixtures thereof as well as fluoropolymers such as pvdf and ptfe.

In one set of experiments, polymer cartridges of 40 micron porosity and an average weight of 17.4±-mg were prepared by sintering different HMWPE/CPG mixtures in a mold at 170 C. The mold size was chosen to produce cartridges of a diameter of 4 mm and a height of 3.4 mm, which fit into standard synthesis columns used for high through-put synthesis in 96-well plate format. 1018-HMWPE was doped with T, dA, dC and dG-CPG of 1000 Angstrom pore size and 35 umol/g loading. Of each type of CPG 4.3 mg were incorporated, resulting in cartridges of 25% CPG-content by weight and a synthesis scale of 150 nmol. A second set of cartridges was prepared by incorporating 5.7 mg of each type of CPG corresponding to 33% CPG-content and a synthesis scale of 200 nmol. These cartridges will be referred to as 25%- and 33%-cartidges. Synthesis tests were carried out using the HMWPE-cartridges containing 25% of dT-CPG, which corresponds to a 150 nmol synthesis scale. The sequence assembly was carried out under standard synthesis conditions and reagent consumption for a 150 mnol synthesis scale. A set of 4 sequences with increasing length, a 20 mer, 30 mer, 40 mer and 50 mer, was synthesized in accordance with Table 2 below:

| | |
|---|---|
| Sequence | 5′ - 3′ |

| | |
|---|---|
| 20 mer | ACGTACGTACGTACGTACGTACGT |
| 30 mer | ACGTACGTACGTACGTACGTACGTACGTACGTAT |
| 40 mer | ACGTACGTACGTACGTACGTACGTACGTACGTACGTACGT |
| 50 mer | ACGTACGTACGTACGTACGTACGTACGTACGTACGTACGTACGTACGTAT |

The cartridges were next subjected to standard methylamine deprotection and the oligonucleotides were eluted from the column in 500 ul of water. The obtained yields in optical density (OD) values and the corresponding total umol are summarized in Table 3:

| Sequence | total OD | ε [L mol⁻¹ cm⁻¹] | Crude nmol |
|---|---|---|---|
| 20mer | 10 | 104100 | 100 |
| 30mer | 8 | 134800 | 60 |
| 40mer | 14 | 184100 | 76 |
| 50mer | 11.5 | 214800 | 53 |

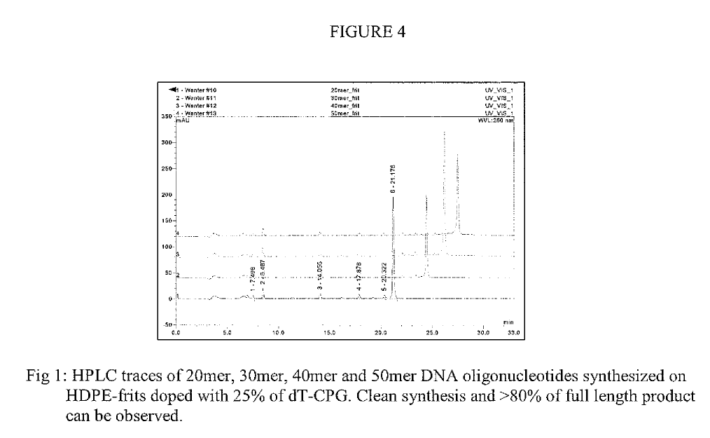

The oligonucleotides were analyzed by ion exchange HPLC, giving detailed information about their synthesis quality and purity. The chromatograms of all four syntheses are shown in FIG. 4. They all show good coupling efficiency throughout the synthesis and the content of full length product is >80%.

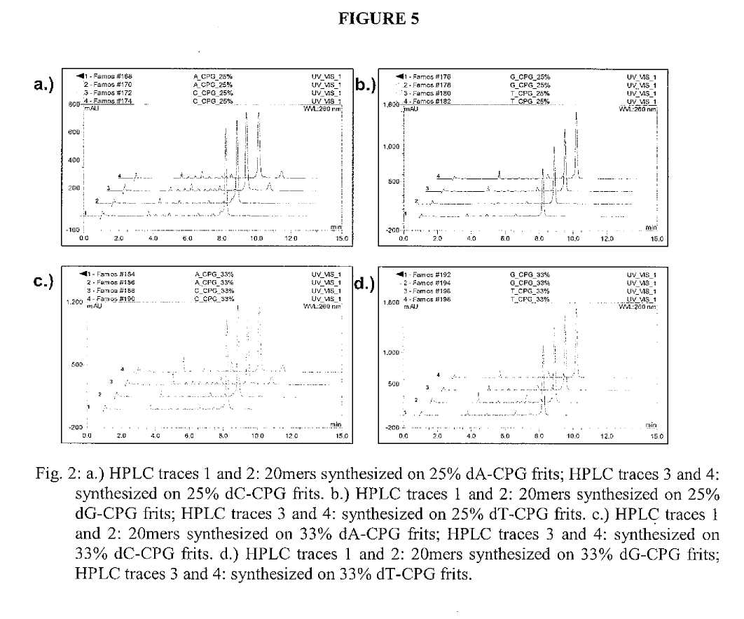

In a second experiment, HMWPE cartridges containing either 25% (150 nmol scale) or 33% (200 nmol scale) of derivatized CPG were prepared for dA-, dC- and dG-support as well. 20 mer DNA sequences were synthesized on these cartridges. As shown in FIG. 5, quantification showed consistent ODs and HPLC-analysis confined good coupling efficiency for all types of solid support. The measured total ODs and nmol were all in the range of a standard 150 nmol synthesis scale. The table below gives the average values of eight synthesis performed for each type of solid support.

| Support | % CPG | Av. Toatal ODs | ε [L mol⁻¹ cm⁻¹] | Crude nmol |
|---|---|---|---|---|
| dA | 25% | 8.5 | 207041 | 40.8 |
| dC | 25% | 9.6 | 199796 | 47.8 |
| dG | 25% | 8.4 | 203730 | 41.2 |
| dT | 25% | 10.2 | 201140 | 50.7 |
| dA | 33% | 10.9 | 207041 | 52.7 |
| dC | 33% | 11.2 | 199796 | 55.8 |
| dG | 33% | 10.8 | 203730 | 52.9 |
| dT | 33% | 9.0 | 201140 | 44.9 |

HMWPE cartridges containing 25% or 33% of CPG were successfully employed for the synthesis of DNA oligonucleotides of variable length and variable 3′-end nucleotide. The synthesis quality and yields are comparable to standard 150 nmol columns containing loose CPG. The CPG doped HDPE frits are easier to prepare and to handle than CPG columns containing loose CPG. The dead volume is reduced by approximately 50% with respect to frit-CPG-frit sandwich of existing synthesis column technology. The possibility of preparing these cartridges with lower CPG-content than 25% or alternatively, same CPG-content but smaller dimensions, makes the present disclosure ideal to reduce the synthesis scale below the current limit of 10 nmol.

Although an embodiment of the disclosure has been described using specific terms and devices, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of various other embodiments may be interchanged both in whole or in part. Therefore, the spirit and scope of the appended claims should not be limited to the description of the preferred version contained herein.

We claim:

1. A method of synthesizing oligonucleotides of a predetermined sequence, the method comprising the steps of:
a. providing a synthesis column;
b. providing a polymer cartridge containing variable amounts of a solid support embedded within said cartridge, and derivatized with the first nucleotide of said sequence, disposed within said column; and
c. introducing at least one reagent to said column to add the subsequent nucleotide of said predetermined sequence and

2. The method of claim I where said solid support is Controlled Porous Glass.

3. The method of claim 1 where said solid support is cross-linked polystyrene.

4. The method of claim 1 further comprising the step of repeating step (c) until the oligonucleotide of the predetermined sequence is obtained.

5. The method of claim 4 further comprising the step of cleaving the oligonucleotide from said cartridge.

6. The method of claim 5 further comprising the step of purifying said oligonucleotide.

7. A method of synthesizing oligonucleotides of a predetermined sequence, the method comprising the steps of:
a. providing a synthesis column;
b. providing a polymer cartridge containing variable amounts of a solid support embedded within said cartridge, and derivatized with the first nucleotide of said sequence, disposed within said column;
c. performing the Phosphoramidite method for the stepwise addition of the subsequent nucleotide in the predetermined sequence; and
d. repeating step (c) until the oligonucleotide of the predetermined sequence is obtained.

8. The method of claim 7 where said solid support is controlled porous Glass.

9. The method of claim 7 where said solid support is cross-linked Polystyrene.

10. The method of claim 7 further comprising the step of cleaving the oligonucleotide from said cartridge.

11. The method of claim 10 further comprising the step of purifying said oligonucleotide.

12. The method of claim 7 wherein said Phosphoramidite method includes the steps of releasing the 5′-OH of said first nucleotide, coupling said subsequent nucleotide in its phosphoramidite form, capping the unreacted 5′-OH position of said subsequent nucleotide, and oxidizing said subsequent nucleotide.

13. A method of synthesizing oligonucleotides of a predetermined sequence, the method comprising the steps of:
a. providing a synthesis column;
b. providing a polymer cartridge containing variable amounts of a solid support embedded within said cartridge, and derivatized with the first nucleotide of said sequence, disposed within said column;
c. introducing at least one reagent to said column to add the subsequent nucleotide of said predetermined sequence;

14. The method of claim 13 where said solid support is Controlled Porous Glass.

15. The method of claim 13 where said solid support is cross-linked Polystyrene.

16. The method of claim 13 further comprising the step of repeating step (c) until the oligonucleotide of the predetermined sequence is obtained.

17. The method of claim 16 further comprising the step of cleaving the oligonucleotide from said cartridge.

18. The method of claim 17 further comprising the step of purifying said oligonucleotide.

19. A method of synthesizing oligonucleotides of a predetermined sequence, the method comprising the steps of:
a. providing a synthesis column;
b. providing a polymer cartridge containing variable amounts of a solid support embedded within said cartridge, and derivatized with a universal linker suitable for oligonucleotide synthesis, disposed within said column; and
c. introducing at least one reagent to said column to add the subsequent nucleotide of said predetermined sequence and

20. The method of claim 19 where said solid support is Controlled Porous Glass.

21. The method of claim 19 where said solid support is cross-linked polystyrene.

22. The method of claim 19 further comprising the step of repeating step (c) until the oligonucleotide of the predetermined sequence is obtained.

23. The method of claim 22 further comprising the step of cleaving the oligonucleotide from said cartridge.

24. The method of claim 23 further comprising the step of purifying said oligonucleotide.
